(19) United States
(12) Patent Application Publication
(10) Pub. No.: US 2025/0127516 A1
(43) Pub. Date: Apr. 24, 2025

(54) MULTI-LUMEN CATHETER

(71) Applicant: ABIOMED, Inc., Danvers, MA (US)

(72) Inventors: Anthony Wong, Danvers, MA (US); Matthew Jenkins, Danvers, MA (US)

(73) Assignee: ABIOMED, Inc., Danvers, MA (US)

(21) Appl. No.: 18/923,012

(22) Filed: Oct. 22, 2024

Related U.S. Application Data
(60) Provisional application No. 63/545,286, filed on Oct. 23, 2023.

(51) Int. Cl. A61B 17/12; A61B 17/00; A61B 90/00

(52) U.S. Cl. CPC A61B 17/12036; A61B 17/00234; A61B 17/12045; A61B 90/06; A61B 2017/00022; A61B 2017/00305; A61B 2017/00557; A61B 2017/00561; A61B 2017/00862; A61B 2090/064

(57) ABSTRACT

Disclosed are devices and systems utilizing specifically designed catheters. The catheters may include a tubular body having a first end and a second end. The tubular body may have an outer diameter that is no more than 9 Fr. The tubular body may define a plurality of lumen extending at least partially through the catheter (e.g., from the first end towards the second end). A first lumen may be configured to slidably receive a guidewire. A second lumen may be configured to inflate and/or deflate a first expandable occlusion element. A third lumen may be configured to inflate and/or deflate a second expandable occlusion element. The catheter may include a plurality of additional lumen.

### CROSS-REFERENCE TO RELATED APPLICATIONS

The present application claims priority to U.S. provisional patent applications 63/545,286 Filed Oct. 23, 2023, which is incorporated by reference herein by its entirety.

### TECHNICAL FIELD

The present disclosure is drawn to multi-lumen catheters, and specifically, to multi-lumen catheters used on devices for cardiovascular treatments.

### BACKGROUND

Devices for circulatory support (e.g., cardiovascular treatment) can be complex, needing to provide multiple functionalities, while also satisfying biological or physical constraints of a patient's body, regulatory requirements, and manufacturability requirements.

If the pumping function of a patient's heart is insufficient despite other medical treatments, the circulatory system can be assisted in various ways. In some cases, a flow-restricting device (which may include, e.g., an inflatable balloon) may be installed into the patient's superior vena cava (SVC) to assist in regulating venous blood return to the heart. By adjusting the flow-restrictive device, the amount of blood flowing back to the heart can be controlled.

### BRIEF SUMMARY

In various aspects, a catheter may be provided. The catheter may include a tubular body. The tubular body may have a first end and a second end. The tubular body may define a plurality of lumens. Each of the plurality of lumen may be separated by an internal wall. The plurality of lumen may extend partially through the catheter.

The plurality of lumen may include a first lumen. The first lumen may be configured to slidably receive a guidewire. The plurality of lumen may include a second lumen. The second lumen may be configured to inflate and/or deflate a first expandable occlusion element. The plurality of lumen may include a third lumen. The third lumen may be configured to inflate and/or deflate a second expandable occlusion element. The plurality of lumen may include a plurality of additional lumen.

In some embodiments, an outer diameter of the tubular body may be no more than 9 Fr.

In some embodiments, each lumen may have a different cross-sectional area. In some embodiments, at least two lumen may have a same cross-sectional area.

In some embodiments, the plurality of lumen includes five lumen. The plurality of lumen may include a six lumen.

In some embodiments, the first lumen may have a circular cross-section. In some embodiments, only the first lumen may have a regular-shaped geometric cross-section. In some embodiments, the total cross-sectional area of material defining the tubular body may be less than 2.5 mm². In some embodiments, the average thickness of the internal wall separating one lumen from an adjacent lumen may be less than 0.2 mm. In some embodiments, the average thickness of the internal wall may be less than 0.13 mm. In some embodiments, the average thickness of each internal wall may be between 0.1 mm-0.2 mm. In some embodiments, the average thickness of one internal wall may be different from an average thickness of at least one other internal wall. In some embodiments, the average thickness of one internal wall may be identical to an average thickness of at least one other internal wall.

In some embodiments, the outer diameter of the tubular body may be no more than 7 Fr.

In some embodiments at least one lumen of the plurality of additional lumen may be configured to terminate on an external surface of the catheter at an intermediate location between the first end and the second end of the tubular body. The at least one lumen of the plurality of additional lumen may be configured to transmit a fluid to the intermediate location. The at least one lumen of the plurality of additional lumen may be configured to draw fluid from the intermediate location.

In some embodiments, at least one of the plurality of additional lumen may define a fluid-filled pressure transducer. In some embodiments, the second lumen may not be adjacent to any lumen defining a fluid-filled pressure transducer. In some embodiments, the third lumen may not be adjacent to any lumen coupled to or defining a fluid-filled pressure transducer.

In some embodiments, the catheter may be free of a lumen having a central axis that is coaxial with a central axis of the catheter. In some embodiments, at least one of the plurality of additional lumen may define a vacuum lumen. The vacuum lumen may be operably coupled to the first expandable occlusion element or the second expandable occlusion element. The vacuum lumen may not be adjacent to any lumen coupled to or defining a fluid-filled pressure transducer.

In various aspects, a system may be provided. The system may include a catheter as disclosed herein. The system may include a first expandable occlusion element operably coupled to the second lumen. The system may further include a second expandable occlusion element operably coupled to the third lumen.

In some embodiments, the first expandable occlusion element may be a balloon. In some embodiments, the second expandable occlusion element may be a balloon. The second expandable occlusion element may have an expanded maximum diameter that may be different from an expanded maximum diameter of the first expandable occlusion element. The first expandable occlusion element may be coupled to an external surface of the catheter at an intermediate point between the first end and the second end of the catheter. The second expandable occlusion element may be coupled to an external surface of the catheter at the second end of the catheter.

In some embodiments, the system may further include a controller. The controller may be operably coupled to the first end of the catheter.

### BRIEF DESCRIPTION OF FIGURES

The accompanying drawings, which are incorporated in and constitute a part of this specification, illustrate embodiments of the present invention and, together with a general description of the invention given above, and the detailed description of the embodiments given below, serve to explain the principles of the present invention.

FIG. 1 is an illustration showing a device with two expandable occlusion elements within a patient.

FIG. 2 is a cross-sectional view of a catheter of the device in FIG. 1.

FIGS. 3A and 3B are illustrations of perspective views of a portion of different embodiments containing a first expandable occlusion element.

FIGS. 4A and 4B are graphs of pressure over time for right atrial pressure and pressure of an occlusion element.

FIG. 5 is an illustration of a system.

It should be understood that the appended drawings are not necessarily to scale, presenting a somewhat simplified representation of various features illustrative of the basic principles of the invention. The specific design features of the sequence of operations as disclosed herein, including, for example, specific dimensions, orientations, locations, and shapes of various illustrated components, will be determined in part by the particular intended application and use environment. Certain features of the illustrated embodiments have been enlarged or distorted relative to others to facilitate visualization and clear understanding. In particular, thin features may be thickened, for example, for clarity or illustration.

### DETAILED DESCRIPTION

The following description and drawings merely illustrate the principles of the invention. It will thus be appreciated that those skilled in the art will be able to devise various arrangements that, although not explicitly described or shown herein, embody the principles of the invention and are included within its scope. Furthermore, all examples recited herein are principally intended expressly to be only for illustrative purposes to aid the reader in understanding the principles of the invention and the concepts contributed by the inventor(s) to furthering the art and are to be construed as being without limitation to such specifically recited examples and conditions. Additionally, the term, “or,” as used herein, refers to a non-exclusive or, unless otherwise indicated (e.g., “or else” or “or in the alternative”). Also, the various embodiments described herein are not necessarily mutually exclusive, as some embodiments can be combined with one or more other embodiments to form new embodiments.

The numerous innovative teachings of the present application will be described with particular reference to the presently preferred exemplary embodiments. However, it should be understood that this class of embodiments provides only a few examples of the many advantageous uses of the innovative teachings herein. In general, statements made in the specification of the present application do not necessarily limit any of the various claimed inventions. Moreover, some statements may apply to some inventive features but not to others. Those skilled in the art and informed by the teachings herein will realize that the invention is also applicable to various other technical areas or embodiments.

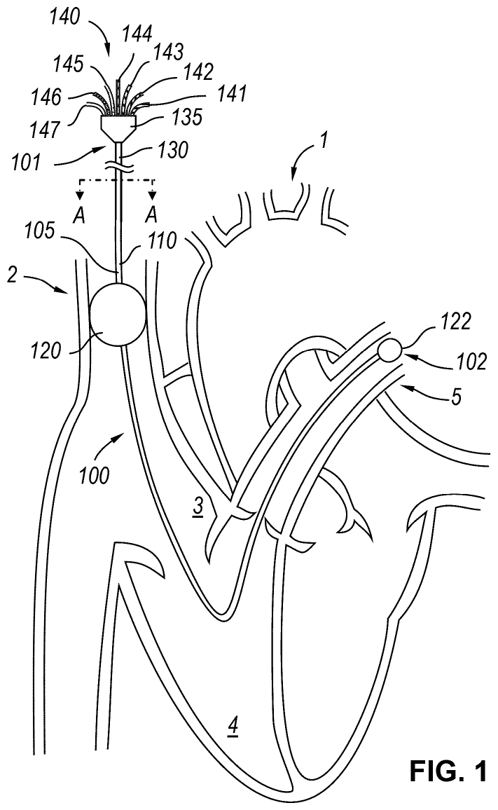

As seen in FIG. 1, in some cardiovascular treatments, a device 100 may be deployed at least partially within a patient. Here, the device is shown as being inserted into a patient's heart 1, extending through the superior vena cava 2, right atrium 3, right ventricle 4, and into the pulmonary artery 5. The device is shown as including a catheter 105. The catheter may have a tubular body 110 with a first end 101 and a second end 102. The device is shown as having two expandable occlusion elements coupled to the catheter-a first expandable occlusion element 120 (shown here deployed in the superior vena cava, at an intermediate point on the catheter between a first end 101 and second end 102 of the catheter), and a second expandable occlusion element 122 (which here is deployed in the pulmonary artery, at a distal end of the catheter). As will be appreciated, the first expandable occlusion element may be located at any place inside of the patient. There may be a portion 130 of the catheter that extends outside the patient's body (e.g., at an access site). As shown, to control the device, there may need to be a number of connections 140 to the catheter. Here, we see multiple connections, which may include, e.g., tubes 141, 142 (e.g., cannulas) for connecting a fluid source (not shown) to the expandable occlusion elements, a tube 143 (e.g., cannula) for drawing fluid from the patient out through the catheter, a guidewire 144, wires or tubes 145, 146 (e.g., cannulas) for connecting to sensors in or on the device, and a tube 147 (e.g., cannula) for a vacuum to be applied to one or both of the expandable occlusion elements. As will be appreciated, any suitable number of connections may be used to connect to the catheter.

Ensuring all of the necessary connections can be made through the catheter can be challenging, especially when the catheter diameter is small.

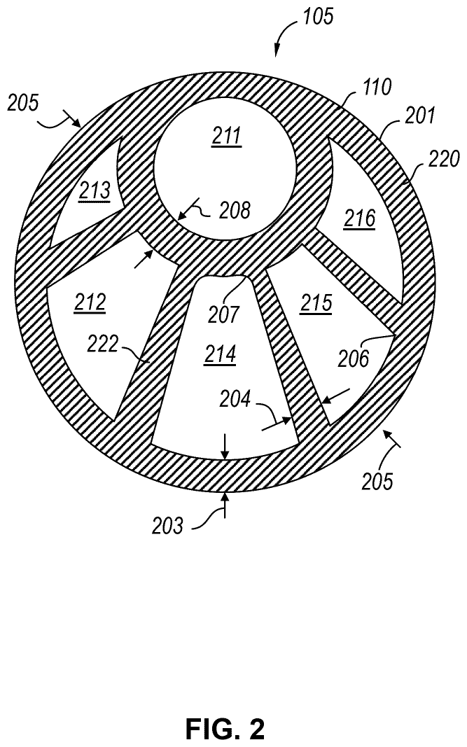

Referring to FIG. 2, a cross-section of the catheter based on cutting plane A in FIG. 1 can be seen. The tubular body 110 may have an external surface 201 defined by an external wall 220. An outer diameter 205 of the tubular body may be no more than 9 Fr. In some embodiments, an outer diameter of the tubular body may be no more than 7 Fr. As used herein, the term “outer diameter” refers to the maximum distance between two points on the external surface of the tubular body; e.g., if the tubular body has an oval cross-section, the outer diameter will be the length of the major axis of the oval. In some embodiments, the external surface may have a circular cross-section. In some embodiments, the external surface may have an oval cross-section. In some embodiments, the external surface may have a freeform or irregular-shaped cross-section.

The tubular body may define a plurality of lumen extending at least partially through the catheter. For purposes herein, extending at least partially through the catheter may mean that each lumen may include a cross-sectional area less than a cross sectional area of the catheter. It also may mean that the lumen need not extend along an entire length of the catheter.

In some embodiments, the plurality of lumens may include five or more lumen. For example, in some embodiments, there may be five lumens. In some embodiments, there may be six lumens. A first lumen 211 may be configured to slidably receive a guidewire. A second lumen 212 may be configured to inflate and/or deflate a first expandable occlusion element. A third lumen 213 may be configured to inflate and/or deflate a second expandable occlusion element. The catheter may include a plurality of additional lumen (e.g., fourth lumen 214, fifth lumen 215, and sixth lumen 216).

Referring to FIG. 2, in some embodiments, the first lumen 211 may be configured to slidably receive a guidewire/PA. In some embodiments the fifth lumen 215 and sixth lumen may be configured to measure a right atrial pressure and/or a jugular venous pressure. The sixth lumen 216 may be configured to receive a fluid filled pressure sensor. The fifth lumen 215 may also be configured to receive a fluid filled pressure sensor. In some embodiments, the fourth lumen 214 may be configured to inflate and/or deflate a second expandable occlusion element, or (preferably) as an infusion or blood draw line. The third lumen 213 may be configured to inflate and/or deflate an expandable occlusion element. The second lumen 213 may be configured to inflate and/or deflate an expandable occlusion element. In some embodiments, the arrangement of the pressure sensors, and the inflation/deflation lumens are arranged to limit the amount of crosstalk between the lumens. In some embodiments, the lumens configured to measure right atrial pressure and jugular venous pressure are not adjacent to the lumen including the floatation element. This arrangement may assist in reducing the amount of crosstalk between the pressure sensors and the floatation element.

In some embodiments, the pressure sensor may be a fluid filled pressure transducer. The fluid filled pressure transducer “translates” pressure from the lumen into a pressure value that can be output to an external system or display. In some embodiments, the pressure sensor may be an optical pressure sensor.

The cross-sectional area of each lumen may vary as needed based on the specific requirements of the device. In some embodiments, each lumen may have a cross-sectional area between 0.1 mm² and 0.5 mm². In some embodiments, each lumen may have a cross-sectional area between 0.125 mm² and 0.475 mm². Each lumen may have a different cross-sectional area. In some embodiments, at least two lumens may have a same cross-sectional area. The first lumen may have a circular cross-section. In some embodiments, only the first lumen may have a regular-shaped geometric cross-section. In some embodiments, one or more angled corners 206 may be present, formed at the connection of an internal wall 222 and an external wall 220, or between two internal walls. In some embodiments, one or more rounded corners 207 may be present, formed at the connection of an internal wall 222 and an external wall 220, or between two internal walls. In some embodiments, each lumen may be free of angled corners.

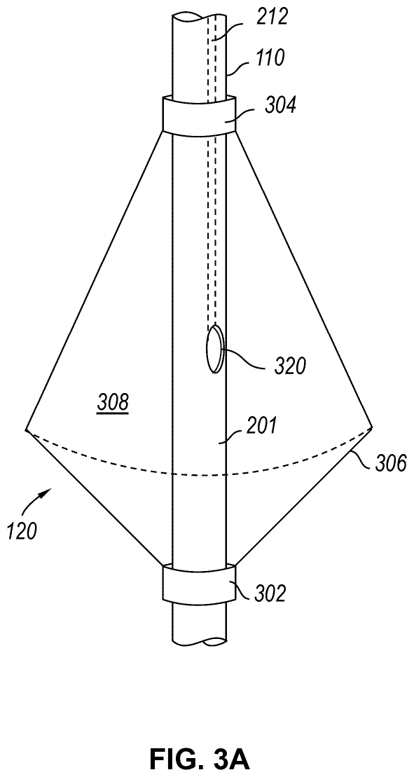

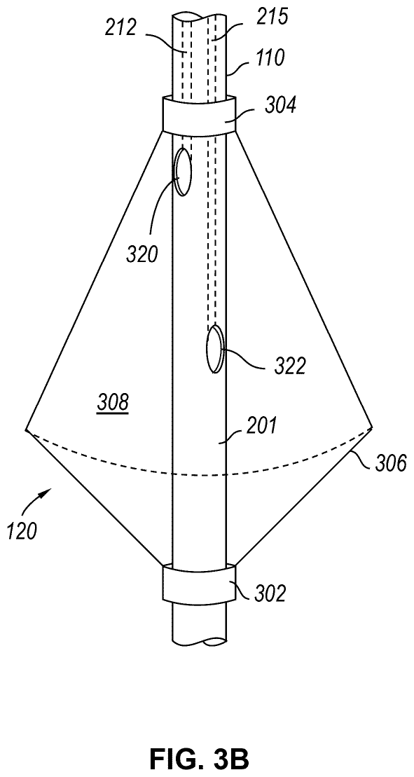

Referring briefly to FIGS. 3A and 3B, one or more lumen may extend only partially through the catheter. In some embodiments, the lumen may exit the catheter at one or more exit points on an external surface of the catheter. In FIG. 3A, a fill/vacuum lumen (e.g., second lumen 212) exits through the catheter at a first location 320. The first expandable occlusion element 120 is disposed around the exit point. The first expandable occlusion element is shown here as being coupled (e.g., bonded) to the catheter at a first end 302 and a second end 304, where an intermediate part 306 of the first expandable occlusion element is the part that can expand as fluid (such as air or a saline fluid, which may include a contrast agent) is provided to (e.g., via second lumen 212 and exit point 320) the volume of space 308 between the intermediate part and the catheter.

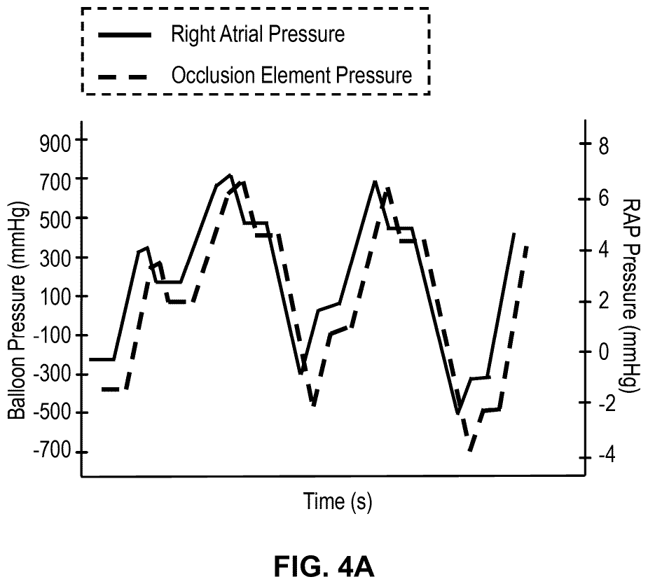

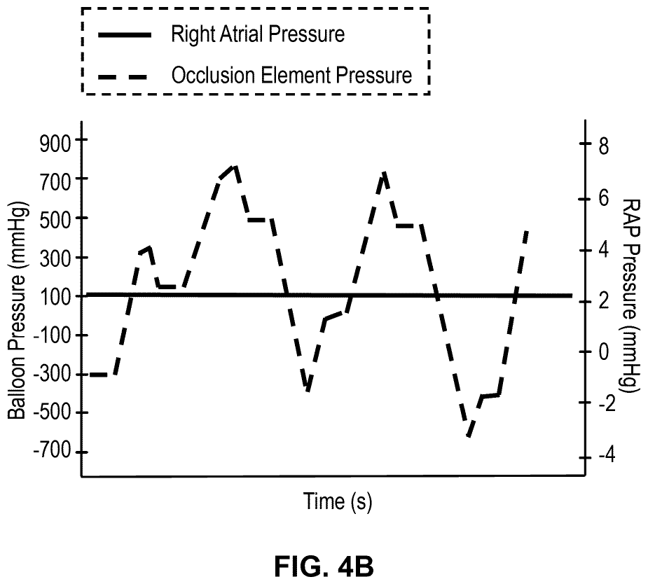

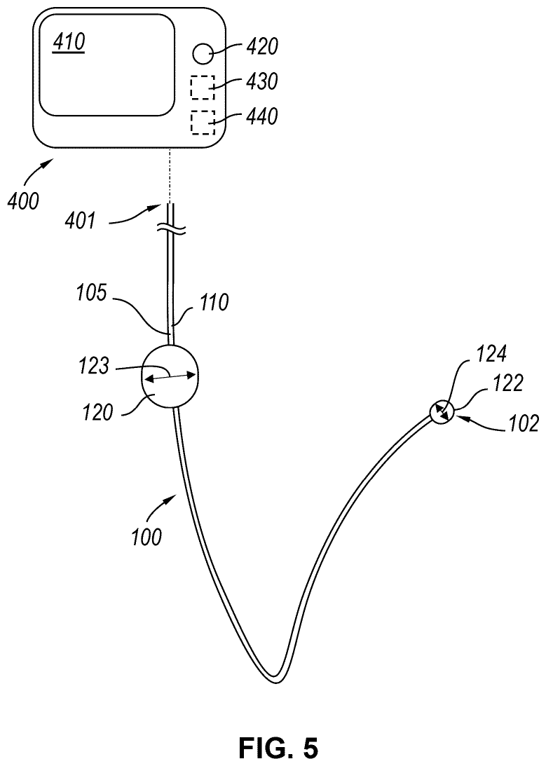

In FIG. 3B, a fill lumen (e.g., second lumen 212) is still shown as having an exit point 320 at a first location. In some embodiments, at least one of the additional lumens (here, fifth lumen 215) may be configured to terminate on an external surface 201 of the catheter at an intermediate location (here, exit point 322) between the first end and the second end of the tubular body. In some embodiments, such lumen may connect to a sensor at or on the surface of the catheter. In some embodiments, such lumen may be configured to transmit a fluid to the intermediate location, or draw fluid from the intermediate location. In some embodiments, the intermediate location may be within the first expandable occlusion element (as seen in FIG. 4). In some embodiments, the intermediate location may be within the second expandable occlusion element. In some embodiments, the intermediate location may be between the first and second expandable occlusion elements.

In various embodiments, at least one of the additional lumens may define a fluid-filled pressure transducer. Such techniques are well-understood in the art.

As catheters become smaller and smaller, the internal walls of the catheter may also become thinner and thinner. As the internal walls become thinner, they may become more flexible. As such, the use of one lumen may become more influential with regards to the use of adjacent lumens. For example, in FIG. 2, if fifth lumen 215 is used to pass blood through the lumen in a pulsatile fashion, using fourth lumen 214 as a fluid-filled pressure transducer may be challenging, as there may be “crosstalk” between the two lumens. In some instances, depending on the exact design and use of the lumen, this may or may not be an issue.

To minimize the risk of crosstalk for certain lumen, in some embodiments, the second lumen may not be adjacent to any lumen defining a fluid-filled pressure transducer. In some embodiments, the third lumen may not be adjacent to any lumen coupled to or defining a fluid-filled pressure transducer. In some embodiments, neither the second nor the third lumen are adjacent to any lumen coupled to or defining a fluid-filled pressure transducer.

Referring to FIGS. 4A and 4B, graphs of pressure over time are shown. The graphs display a measured pressure of a lumen coupled to an expandable occlusion element, and a lumen configured to measure right atrial pressure. In FIG. 4A, the measured right atrial pressure and expandable occlusion element pressure were measured in an arrangement where the right atrial pressure lumen is adjacent to the expandable occlusion element lumen. In FIG. 4A, the measured right atrial pressure follows the trend of the occlusion element pressure. The trend of the right atrial pressure is indicative of crosstalk between lumen. In FIG. 4B, the right atrial pressure and the occlusion element pressure were measured in an arrangement where the right atrial pressure and expandable occlusion element were not adjacent. In FIG. 4B, the right atrial pressure does not follow the trend of the occlusion element pressure. The trend displayed in FIG. 4B indicates that there was no crosstalk between the two lumen.

In some embodiments, at least one of the additional lumens may be configured to receive one or more optical fibers. In some embodiments, the catheter may include one or more optical fibers. In some embodiments, the catheter may include a plurality of optical fibers. In some embodiments, there may be one optical fiber in a single lumen. In some embodiments, there may be a plurality of optical fibers in a single lumen.

At least one of the additional lumens may define a vacuum lumen operably coupled to the first expandable occlusion element or the second expandable occlusion element. In FIG. 3B, a fifth lumen 215 is shown as having an exit point 322, the exit point being the point through which fluid (such as air) is added and/or removed from the first expandable occlusion element. As drawing vacuum may cause internal walls to flex, in some embodiments, the vacuum lumen may not be adjacent to any lumen coupled to or defining a fluid-filled pressure transducer.

Referring to FIG. 2, a cross-section of the catheter can be seen where the lumens are neither symmetrically nor concentrically arranged. In some embodiments, the arrangement of lumen within the tubular body may be bilaterally symmetrical. In some embodiments, the catheter may be free of a lumen having a central axis that is coaxial with a central axis of the catheter (e.g., a central axis of first lumen 211 is not coaxial with a central axis defining tubular body 110, same with second lumen 212, etc.). Although, as will be appreciated in view of FIG. 2, the central axis of one or more lumen may be parallel to the central axis of the catheter.

A total cross-sectional area of material defining the tubular body (including external walls and internal walls defining the various lumen) may be no more than 3 mm². A total cross-sectional area of material defining the tubular body may be no more than 2.9 mm². A total cross-sectional area of material defining the tubular body may be no more than 2.8 mm². A total cross-sectional area of material defining the tubular body may be no more than 2.7 mm². A total cross-sectional area of material defining the tubular body may be no more than 2.6 mm². A total cross-sectional area of material defining the tubular body may be no more than 2.5 mm². A total cross-sectional area of material defining the tubular body may be no more than 2.4 mm². A total cross-sectional area of material defining the tubular body may be at least 2.3 mm². A total cross-sectional area of material defining the tubular body may be at least 2.2 mm². A total cross-sectional area of material defining the tubular body may be at least 2.1 mm². A total cross-sectional area of material defining the tubular body may be at least 2 mm².

In some embodiments, an average thickness 204 of an internal wall 222 separating one lumen from an adjacent lumen may be less than 0.2 mm. In some embodiments, an average thickness of an internal wall separating one lumen from an adjacent lumen may be less than 0.19 mm. In some embodiments, an average thickness of an internal wall separating one lumen from an adjacent lumen may be less than 0.18 mm. In some embodiments, an average thickness of an internal wall separating one lumen from an adjacent lumen may be less than 0.17 mm. In some embodiments, an average thickness of an internal wall separating one lumen from an adjacent lumen may be less than 0.16 mm. In some embodiments, an average thickness of an internal wall separating one lumen from an adjacent lumen may be less than 0.15 mm. In some embodiments, an average thickness of an internal wall separating one lumen from an adjacent lumen may be less than 0.14 mm. In some embodiments, an average thickness of an internal wall separating one lumen from an adjacent lumen may be less than 0.13 mm. In some embodiments, an average thickness of an internal wall separating one lumen from an adjacent lumen may be at least 0.1 mm. In some embodiments, an average thickness of each internal wall may be 0.1 mm-0.2 mm.

In some embodiments, an average thickness of one internal wall may be different from an average thickness of at least one other internal wall. An average thickness of one internal wall may be identical to an average thickness of at least one other internal wall. In some embodiments, an average thickness 208 of an internal wall separating the first lumen from any adjacent lumen may be thicker than an average thickness 204 of any other internal wall. In some embodiments, an average thickness 208 of an internal wall separating the first lumen from any adjacent lumen may be thicker than an average thickness 204 of an internal wall separating a first additional lumen from an adjacent additional lumen. In some embodiments, an average thickness 208 of an internal wall separating the first lumen from any adjacent lumen may be thicker than an average thickness 203 of an external wall.

In some embodiments, the tubular body may be a polyurethane material. In some embodiments, the polyurethane material may be Pellethane 69D. In some embodiments, the polyurethane material may be PVC.

In various aspects a system may be provided. Referring to FIGS. 1 and 2, the system may include an embodiment of a catheter 105 as disclosed herein. The system may include a first expandable occlusion element 120, which may be operably coupled to the second lumen 212 of the catheter. The system may include a second expandable occlusion element 122, which may be operably coupled to the third lumen 213 of the catheter. The first expandable occlusion element may be a balloon. The second expandable occlusion element may be a balloon. Referring to FIG. 4, the second expandable occlusion element may have an expanded maximum diameter 124 that is different from an expanded maximum diameter 123 of the first expandable occlusion element. The expanded maximum diameter 123 of the first expandable occlusion element may be larger than the expanded maximum diameter 124 of the second expandable occlusion element. The first expandable occlusion element may be coupled to an external surface of the catheter at an intermediate point between the first end and the second end of the catheter. The second expandable occlusion element may be coupled to an external surface of the catheter at the second end of the catheter.

The system may include a controller 400 (see FIG. 4) operably coupled to the first end 401 of the catheter. The controller may include one or more displays 410 (which may be, e.g., touch-screen displays), one or more knobs or buttons 420, one or more processors 430, and one or more non-transitory computer-readable storage devices 440, which contain instructions that, when executed by the processor(s), cause the processor(s) to operate the device as designed. For example, the processor(s) may be configured to, collectively, cause the device to selectively occlude, e.g., the superior vena cava via inflating the first balloon for a period of time, and then deflating. Optionally, this could include repeating the inflating/deflating process at a predetermined interval.

Various modifications may be made to the systems, methods, apparatus, mechanisms, techniques, and portions thereof described herein with respect to the various figures, such modifications being contemplated as being within the scope of the invention. For example, while a specific order of steps or arrangement of functional elements is presented in the various embodiments described herein, various other orders/arrangements of steps or functional elements may be utilized within the context of the various embodiments. Further, while modifications to embodiments may be discussed individually, various embodiments may use multiple modifications contemporaneously or in sequence, compound modifications and the like.

Although various embodiments which incorporate the teachings of the present invention have been shown and described in detail herein, those skilled in the art can readily devise many other varied embodiments that still incorporate these teachings. Thus, while the foregoing is directed to various embodiments of the present invention, other and further embodiments of the invention may be devised without departing from the basic scope thereof. As such, the appropriate scope of the invention is to be determined according to the claims.

1. A catheter, comprising:
a tubular body having a first end and a second end, the tubular body defining a plurality of lumen extending at least partially through the catheter; and
wherein the plurality of lumen include:
a first lumen is configured to slidably receive a guidewire;
a second lumen is configured to inflate and/or deflate a first expandable occlusion element;
a third lumen is configured to inflate and/or deflate a second expandable occlusion element; and
a plurality of additional lumen;
wherein an outer diameter of the tubular body is no more than 9 Fr.

2. The catheter of claim 1, wherein each lumen has a different cross-sectional area.

3. The catheter of claim 1, wherein at least two lumen have a same cross-sectional area.

4. The catheter of claim 1, wherein the plurality of lumen includes a fifth lumen.

5. The catheter of claim 1, wherein the plurality of lumen includes sixth lumen.

6-7. (canceled)

8. The catheter of claim 1, wherein a total cross-sectional area of material defining the tubular body is less than 2.5 mm².

9. The catheter of claim 1, wherein an average thickness of an internal wall separating one lumen from an adjacent lumen is less than 0.2 mm.

10. (canceled)

11. The catheter of claim 1, wherein an average thickness of each internal wall is 0.1 mm-0.2 mm.

12-13. (canceled)

14. The catheter of claim 1, wherein the outer diameter is no more than 7 Fr.

15. The catheter of claim 1, wherein at least one lumen of the plurality of additional lumen is configured to terminate on an external surface of the catheter at an intermediate location between the first end and the second end of the tubular body.

16. The catheter of claim 15, wherein the at least one lumen of the plurality of additional lumen is configured to transmit a fluid to the intermediate location.

17. The catheter of claim 15, wherein the at least one lumen of the plurality of additional lumen is configured to draw fluid from the intermediate location.

18. The catheter of claim 1, wherein at least one of the plurality of additional lumen defines a fluid-filled pressure transducer.

19. The catheter of claim 18, wherein the second lumen is not adjacent to any lumen defining a fluid-filled pressure transducer.

20. The catheter of claim 19, wherein the third lumen is not adjacent to any lumen coupled to or defining a fluid-filled pressure transducer.

21. The catheter of claim 1, wherein at least one of the plurality of additional lumen is configured to receive one or more optical fibers.

22. (canceled)

23. The catheter of claim 1, wherein at least one of the plurality of additional lumen defines a vacuum lumen operably coupled to the first expandable occlusion element or the second expandable occlusion element.

24. (canceled)

25. A system, comprising:
a catheter of claim 1;
a first expandable occlusion element operably coupled to the second lumen; and
a second expandable occlusion element operably coupled to the third lumen.

26-28. (canceled)

29. The system of claim 25, wherein the first expandable occlusion element is coupled to an external surface of the catheter at an intermediate point between the first end and the second end of the catheter.

30. The system of claim 25, wherein the second expandable occlusion element is coupled to an external surface of the catheter at the second end of the catheter.

31. The system of claim 25, further comprising a controller operably coupled to the first end of the catheter.
